SEQUENCE LISTING (13 sequences)

SEQ ID NO: 1 - DNA, 2323 bp, Homo sapiens
ggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcaaaaaaaaaaaaaaaaaa

SEQ ID NO: 2 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 3 - protein, 57 aa, Homo sapiens
DVKCDMEVSCPDGYTCCRLQSGAWGCCPFTQAVCCEDHIHCCPAGFTCDTQKGTCEQ

SEQ ID NO: 4 - protein, 57 aa, Homo sapiens
VMCPDARSRCPDGSTCCELPSGKYGCCPMPNATCCSDHLHCCPQDTVCDLIQSKCLS

SEQ ID NO: 5 - protein, 54 aa, Homo sapiens
VPCDNVSSCPSSDTCCQLTSGEWGCCPIPEAVCCSDHQHCCPQGYTCVAEGQCQ

SEQ ID NO: 6 - protein, 56 aa, Homo sapiens
DIGCDQHTSCPVGQTCCPSLGGSWACCQLPHAVCCEDRQHCCPAGYTCNVKARSCE

SEQ ID NO: 7 - protein, 56 aa, Homo sapiens
DVECGEGHFCHDNQTCCRDNRQGWACCPYRQGVCCADRRHCCPAGFRCAARGTKCL

SEQ ID NO: 8 - protein, 57 aa, Homo sapiens
AIQCPDSQFECPDFSTCCVMVDGSWGCCPMPQASCCEDRVHCCPHGAFCDLVHTRCI

SEQ ID NO: 9 - protein, 56 aa, Homo sapiens
GGPCQVDAHCSAGHSCIFTVSGTSSCCPFPEAVACGDGHHCCPRGFHCSADGRSCF

SEQ ID NO: 10 - protein, 71 aa, Artificial Sequence
  Description of Artificial Sequence Synthetic polypeptide
AMDIGCDQHTSCPVGQTCCPSLGGSWACCQLPHAVCCEDRQHCCPAGYTCNVKARSCEKLAAALEHHHHHH

SEQ ID NO: 11 - protein, 72 aa, Artificial Sequence
  Description of Artificial Sequence Synthetic polypeptide
AMAIQCPDSQFECPDFSTCCVMVDGSWGCCPMPQASCCEDRVHCCPHGAFCDLVHTRCIKLAAALEHHHHHH

SEQ ID NO: 12 - protein, 589 aa, Mus musculus
MWVLMSWLAFAAGLVAGTQCPDGQFCPVACCLDQGGANYSCCNPLLDTWPRITSHHLDGSCQTHGHCPAGYSCLLTVSGTSSCCPFSKGVSCGDGYHCCPQGFHCSADGKSCFQMSDNPLGAVQCPGSQFECPDSATCCIMVDGSWGCCPMPQASCCEDRVHCCPHGASCDLVHTRCVSPTGTHTLLKKFPAQKTNRAVSLPFSVVCPDAKTQCPDDSTCCELPTGKYGCCPMPNAICCSDHLHCCPQDTVCDLIQSKCLSKNYTTDLLTKLPGYPVKEVKCDMEVSCPEGYTCCRLNTGAWGCCPFAKAVCCEDHIHCCPAGFQCHTEKGTCEMGILQVPWMKKVIAPLRLPDPQILKSDTPCDDFTRCPTNNTCCKLNSGDWGCCPIPEAVCCSDNQHCCPQGFTCLAQGYCQKGDTMVAGLEKIPARQTTPLQIGDIGCDQHTSCPVGQTCCPSLKGSWACCQLPHAVCCEDRQHCCPAGYTCNVKARTCEKDVDFIQPPVLLTLGPKVGNVECGEGHFCHDNQTCCKDSAGVWACCPYLKGVCCRDGRHCCPGGFHCSARGTKCLRKKIPRWDMFLRDPVPRPLL

SEQ ID NO: 13 - DNA, 2145 bp, Mus musculus
gagatgcctcccagggagcccggaccccgacgcaggcagaccatgtgggtcctgatgagctggctggccttcgcggcagggctggtagccggaacacagtgtccagatgggcagttctgccctgttgcctgctgccttgaccagggaggagccaactacagctgctgtaaccctcttctggacacatggcctagaataacgagccatcatctagatggctcctgccagacccatggccactgtcctgctggctattcttgtcttctcactgtgtctgggacttccagctgctgcccgttctctaagggtgtgtcttgtggtgatggctaccactgctgcccccagggcttccactgtagtgcagatgggaaatcctgcttccagatgtcagataaccccttgggtgctgtccagtgtcctgggagccagtttgaatgtcctgactctgccacctgctgcattatggttgatggttcgtggggatgttgtcccatgccccaggcctcttgctgtgaagacagagtgcattgctgtccccatggggcctcctgtgacctggttcacacacgatgcgtttcacccacgggcacccacaccctactaaagaagttccctgcacaaaagaccaacagggcagtgtctttgcctttttctgtcgtgtgccctgatgctaagacccagtgtcccgatgattctacctgctgtgagctacccactgggaagtatggctgctgtccaatgcccaatgccatctgctgttccgaccacctgcactgctgcccccaggacactgtatgtgacctgatccagagtaagtgcctatccaagaactacaccacggatctcctgaccaagctgcctggatacccagtgaaggaggtgaagtgcgacatggaggtgagctgccctgaaggatatacctgctgccgcctcaacactggggcctggggctgctgtccatttgccaaggccgtgtgttgtgaggatcacattcattgctgcccggcagggtttcagtgtcacacagagaaaggaacctgcgaaatgggtatcctccaagtaccctggatgaagaaggtcatagcccccctccgcctgccagacccacagatcttgaagagtgatacaccttgtgatgacttcactaggtgtcctacaaacaatacctgctgcaaactcaattctggggactggggctgctgtcccatcccagaggctgtctgctgctcagacaaccagcattgctgccctcagggcttcacatgtctggctcaggggtactgtcagaagggagacacaatggtggctggcctggagaagatacctgcccgccagacaaccccgctccaaattggagatatcggttgtgaccagcataccagctgcccagtagggcaaacctgctgcccaagcctcaagggaagttgggcctgctgccagctgccccatgctgtgtgctgtgaggaccggcagcactgttgcccggccgggtacacctgcaatgtgaaggcgaggacctgtgagaaggatgtcgattttatccagcctcccgtgctcctgaccctcggccctaaggttgggaatgtggagtgtggagaagggcatttctgccatgataaccagacctgttgtaaagacagtgcaggagtctgggcctgctgtccctacctaaagggtgtctgctgtagagatggacgtcactgttgccccggtggcttccactgttcagccaggggaaccaagtgtttgcgaaagaagattcctcgctgggacatgtttttgagggatccggtcccaagaccgctactgtaaggaagggctacagacttaaggaactccacagtcctgggaaccctgttccgagggtacccactactcaggcctccctagcgcctcctcccctaacgtctccccggcctactcatcctgagtcaccctatcaccatgggaggtggagcctcaaactaaaaccttcttttatggaaagaaggctgtggccaaaagccccgtatcaaactgccatttcttccggtttctgtggaccttgtggccaggtgctcttcccgagccacaggtgttctgtgagcttgcttgtgtgtgtgtgcgcgtgtgcgtgtgttgctccaataaagtttgtacactttc

(19) United States
(12) Patent Application Publication
(10) Pub. No.: US 2015/0352185 A1
(43) Pub. Date: Dec. 10, 2015

(54) METHOD FOR INCREASING NEPRILYSIN EXPRESSION AND ACTIVITY

(71) Applicants: Denis G. KAY, Stratford (CA); Jackalina M. VAN KAMPEN, Charlottetown (CA)

(72) Inventors: Denis G. KAY, Stratford (CA); Jackalina M. VAN KAMPEN, Charlottetown (CA)

(21) Appl. No.: 13/885,911

(22) PCT Filed: Nov. 16, 2011
(86) PCT No.: PCT/CA11/01260

§ 371 Date: Sep. 16, 2013

Related U.S. Application Data
(60) Provisional application No. 61/414,412, filed on Nov. 16, 2010.

(51) Int. Cl. A61K 38/18; A61K 9/00; A61K 31/713

(52) U.S. Cl. CPC A61K 38/18; A61K 31/713; A61K 9/0085

(57) ABSTRACT

This invention is directed to methods and compositions for increasing the expression or activity of neprilysin in, for example, the frontal cortex or the entorhinal cortex using a progranulin polypeptide or effector. The present invention is further directed to methods of reducing microglia in the brain of a patient with neurodegenerative disease using a progranulin polypeptide or effector.

### FIELD OF THE INVENTION

This invention is directed to methods and compositions for increasing the expression or activity of neprilysin in, for example, the frontal cortex or the entorhinal cortex using a progranulin polypeptide or effector. The present invention is further directed to methods of reducing microglia in the brain of a patient with neurodegenerative disease using a progranulin polypeptide or effector.

### BACKGROUND AND SUMMARY

Progranulin (PGRN) is a growth factor-like protein that is involved in the regulation of multiple processes including development, wound healing, angiogenesis, growth and maintenance of neuronal cells, and inflammation. Altered PGRN expression has been shown in multiple neurodegenerative diseases, including Creutzfeldt-Jakob disease, motor neuron disease, and Alzheimer's disease. For example, recent studies of the genetic etiology of neurodegenerative diseases have shown that heritable mutations in the PGRN gene may lead to adult-onset neurodegenerative diseases due to reduced neuronal survival.

Selective neuronal cell death is the common hallmark of various neurodegenerative disorders. Sporadic forms of Alzheimer's disease, Parkinson's disease, and Lou Gehrig's disease (amyotrophic lateral sclerosis (ALS)) have been linked to environmental factors that cause neuronal cell death by excitotoxicity, oxidative stress, inhibition of parts of the electron transport chain, cellular and mitochondrial membrane disruption, alterations in cellular organelles, alterations in chromatin, general and specific genotoxic action, and inhibition and/or hyperactivation of cell surface protein receptors and effectors. The experimental and clinical literature supports a potential role for excitotoxins in some forms of neurodegeneration, notably ALS and Alzheimer's disease. In particular, abnormalities in glutamate transport have been linked to ALS and domoic acid, a kainate receptor (i.e., an ionotrophic glutamate receptor) agonist, has been shown to be a causal factor in some forms of memory loss, much like that reported in Alzheimer's disease. Oxidative stress has also been linked to the same disease states.

Currently, there is no cure for ALS, Alzheimer's disease (AD), or Parkinson's disease. Current treatment generally involves efforts by physicians to slow progression of the symptoms and make patients more comfortable. While there are a number of drugs in development and a limited number that are FDA approved for treatment (Riluzole, for ALS; L-dopa for Parkinson's disease; cognitive enhancers, such as Aricept, for AD) these treatments only mask the progression of neurologic disease and may act to marginally prolong the lives of some patients. Thus, there is a significant need for methods and compositions directed to treatment of neurodegenerative diseases.

Several embodiments of the invention are described by the following enumerated clauses:

1. A method for increasing the activity of neprilysin in the frontal cortex or entorhinal cortex of a patient with neurodegenerative disease, the method comprising the step of
administering to the patient a composition comprising a progranulin polypeptide wherein the activity of neprilysin in the frontal cortex or entorhinal cortex of the patient is increased.

2. A method for increasing the expression of neprilysin in the frontal cortex or entorhinal cortex of a patient with neurodegenerative disease, the method comprising the step of
administering to the patient a composition comprising a progranulin polypeptide wherein the expression of neprilysin in the frontal cortex or entorhinal cortex of the patient is increased.

3. A method for increasing the activity of neprilysin in the frontal cortex or entorhinal cortex of a patient with neurodegenerative disease, the method comprising the step of
administering to the patient a composition comprising an effector that modifies progranulin expression wherein the activity of neprilysin in the frontal cortex or entorhinal cortex of the patient is increased.

4. A method for increasing the expression of neprilysin in the frontal cortex or entorhinal cortex of a patient with neurodegenerative disease, the method comprising the step of
administering to the patient a composition comprising an effector that modifies progranulin expression wherein the expression of neprilysin in the frontal cortex or entorhinal cortex of the patient is increased.

5. A method for reducing microglia in the brain of a patient with neurodegenerative disease, the method comprising the step of
administering to the patient a composition comprising a progranulin polypeptide wherein the microglia in the brain of the patient are reduced.

6. A method for reducing microglia in the brain of a patient with neurodegenerative disease, the method comprising the step of
administering to the patient a composition comprising an effector that modifies progranulin expression wherein the microglia in the brain of the patient are reduced.

7. A method for increasing the activity or expression of neprilysin in the brain of an individual without a neurodegenerative disease to prevent the neurodegenerative disease, the method comprising the step of
administering to the individual a composition comprising a progranulin polypeptide wherein the neurodegenerative disease is prevented.

8. A method for increasing the activity or expression of neprilysin in the brain of an individual without a neurodegenerative disease to prevent the neurodegenerative disease, the method comprising the step of
administering to the individual a composition comprising an effector that modifies progranulin expression wherein the neurodegenerative disease is prevented.

9. The method of any one of clauses 1, 2, 5 or 7 wherein the amount of the progranulin polypeptide administered to the patient is in the range of about 1 ng/kg of patient body weight to about 1 mg/kg of patient body weight.

10. The method of clause 9 wherein the amount of the progranulin polypeptide administered to the patient is in the range of about 1 ng/kg of patient body weight to about 500 ng/kg of patient body weight.

11. The method of clause 9 wherein the amount of the progranulin polypeptide administered to the patient is in the range of about 1 ng/kg of patient body weight to about 100 ng/kg of patient body weight.

12. The method of any one of clauses 1, 2, 5, 7 or 9 to 11 wherein the composition comprising the progranulin polypeptide is adapted for parenteral administration.

13. The method of clause 12 wherein the route of parenteral administration is selected from the group consisting of intradermally, subcutaneously, intramuscularly, intraperitoneally, intravenously, intraventricularly, intrathecally, intracerebrally, and intracordally.

14. The method of any one of clauses 3, 4, 6 or 8 wherein the amount of the effector administered to the patient is in the range of about 1 ng/kg of patient body weight to about 1 mg/kg of patient body weight.

15. The method of clause 14 wherein the amount of the effector administered to the patient is in the range of about 1 ng/kg of patient body weight to about 500 ng/kg of patient body weight.

16. The method of clause 14 wherein the amount of the effector administered to the patient is in the range of about 1 ng/kg of patient body weight to about 100 ng/kg of patient body weight.

17. The method of any one of clauses 3, 4, 6, 8 or 14 to 16 wherein the composition comprising the effector is adapted for parenteral administration.

18. The method of clause 17 wherein the route of parenteral administration is selected from the group consisting of intradermally, subcutaneously, intramuscularly, intraperitoneally, intravenously, intraventricularly, intrathecally, intracerebrally, and intracordally.

19. The method of any one of clauses 1 to 4 wherein the neurodegenerative disease is Alzheimer's disease.

20. The method of any one of clauses 5 or 6 wherein the neurodegenerative disease is selected from the group consisting of Parkinson's disease, Alzheimer's disease, and amyotrophic lateral sclerosis.

21. The method of any one of clauses 1, 2, 5 or 7 wherein the progranulin polypeptide has at least 95% homology with SEQ ID NO: 2.

22. The method of any one of clauses 3, 4, 6 or 8 wherein the effector is a vector comprising a nucleic acid with at least 95% homology with SEQ ID NO: 1.

23. The method of any one of clauses 1 to 4 or 7 to 8 wherein the neprilysin reduces plaque burden.

In any of the various embodiments described herein, the following features may be present where applicable, providing additional embodiments of the invention.

For all of the embodiments, any applicable combination of embodiments is also contemplated. Any applicable combination of the above-described embodiments is considered to be in accordance with the invention.

### BRIEF DESCRIPTION OF THE DRAWINGS

FIG. 1 shows that ND-602 elevates hippocampal Progranulin (PGRN) expression. Panel A shows immunofluorescent detection of progranulin in micrographs of coronal sections of hippocampus of Tg2576 mice. Original magnification 20×. Panel B shows densitometric quantitation of progranulin content. Mean+/_S.E.M., **P<0.001, *P<0.05.

FIG. 2 shows that ND-602 alters PGRN content in entorhinal and frontal cortex. Panel A shows statistically significant elevation of PGRN content in entorhinal cortex and Panel B demonstrates a trend towards elevated PGRN in frontal cortex of ND602 treated Tg2576 mice. Mean+/_S.E.M. **P<0.001, *P<0.05.

FIG. 3 shows that ND-602 elevates hippocampal neprilysin expression in Tg2576 mice. Micrographs show hippocampal neprilysin expression following treatment with control vector (Panel A, GFP) or ND-602 (Panel B, PGRN). The chart shows densitometric quantitation of neprilysin expression (Panel C). Mean+/_S.E.M. ** P<0.001, *P<0.05.

FIG. 4 shows that ND-602 increases hippocampal expression of neprilysin in normal C57/Bl6 mice (Panel A). A trend towards increased neprilysin activity is observed in frontal cortex of ND-602 treated mice (Panel B). Quantitation of neprilysin activity per milligram wet tissue weight, mean+/_S.E.M. **P<0.001, *P<0.05.

FIG. 5 shows that ND-602 elevates hippocampal protein content when normalized to milligram wet tissue weight (Panel A), this results in a stable neprilysin activity profile (Panel B) when neprilysin activity is normalized to tissue protein content.

FIG. 6 shows the region of the brain where ND-602 was delivered.

FIG. 7 shows that ND-602 reduces plaque burden in the hippocampus of Tg2576 Mice. Thioflavin S histochemistry to detect plaque is shown in Panel A (original magnification 20×). Densitometric quantitation of Thioflavin S reactive plaque demonstrates a significant reduction in hippocampus (Panel B). Mean+/_S.E.M. **P<0.001, *P<0.05.

FIG. 8 shows that ND-602 alters plaque burden in the entorhinal and frontal cortex of Tg2576 Mice. Densitometric quantitation of Thioflavin S reactive plaque demonstrates a significant reduction in entorhinal cortex (Panel A) and a trend towards reduced plaque burden in frontal cortex (Panel B). Mean+/_S.E.M. *P<0.05.

FIG. 9 shows that ND-602 results in reduced neuroinflammation demonstrated by a reduction in microglial cell number in hippocampus of Tg2576 animals. Micrographs demonstrate decreased ILB4 reactive microglial cells in ND-602 treated Tg2576 animals (Panel A: GFP; Panel B: GFAP; Panel C: ND-602; Panel D: ND-602). Densitometric quantitation of ILB4 reactivity shows significant reduction of microglial cell numbers ipsilateral to ND-602 infusion (Panel E). Mean+/_S.E.M. **P<0.001.

### DETAILED DESCRIPTION OF THE INVENTION

Methods and compositions are provided for increasing the activity or expression of neprilysin in the entorhinal cortex or frontal cortex of a patient with neurodegenerative disease. In one illustrative embodiment, the activity or expression of neprilysin in the entorhinal cortex or frontal cortex of the patient can be increased by administering to the patient a composition comprising a progranulin polypeptide or an effector that modifies progranulin expression, wherein the activity of the neprilysin in the entorhinal cortex or frontal cortex of the patient in increased.

In another embodiment, the progranulin polypeptide or the effector can be administered to an individual without a neurodegenerative disease to prevent development of the neurodegenerative disease. In this embodiment, the expression or activity of neprilysin in the brain of the individual can be increased by administering to the individual a composition comprising a progranulin polypeptide or an effector that modifies progranulin expression. The activity or expression of the neprilysin can be increased in, for example, the entorhinal cortex or frontal cortex of the individual.

In another embodiment, the number of microglia in the brain of a patient with a neurodegenerative disease can be reduced by administering to the patient a composition comprising a progranulin polypeptide or an effector that modifies progranulin expression, wherein the microglia in the brain of the individual are reduced.

In the above described illustrative embodiments, the neurodegenerative disease can be mediated by environmental insult.

As used herein “a progranulin” or “a progranulin polypeptide” refers to a polypeptide selected from a polypeptide of SEQ ID NO. 2, a polypeptide with about 95% homology, about 96%, about 97%, about 98%, about 99% homology with SEQ ID NO. 2; a polypeptide of SEQ ID NO. 12, a polypeptide with about 95% homology, about 96%, about 97%, about 98%, about 99% homology with SEQ ID NO. 12; a polypeptide of SEQ ID NO. 3, a polypeptide with about 95% homology, about 96%, about 97%, about 98%, about 99% homology with SEQ ID NO. 3; a polypeptide of SEQ ID NO. 4, a polypeptide with about 95% homology, about 96%, about 97%, about 98%, about 99% homology with SEQ ID NO. 4; a polypeptide of SEQ ID NO. 5, a polypeptide with about 95% homology, about 96%, about 97%, about 98%, about 99% homology with SEQ ID NO. 5; a polypeptide of SEQ ID NO. 6, a polypeptide with about 95% homology, about 96%, about 97%, about 98%, about 99% homology with SEQ ID NO. 6; a polypeptide of SEQ ID NO. 7, a polypeptide with about 95% homology, about 96%, about 97%, about 98%, about 99% homology with SEQ ID NO. 7; a polypeptide of SEQ ID NO. 8, a polypeptide with about 95% homology, about 96%, about 97%, about 98%, about 99% homology with SEQ ID NO. 8; or a polypeptide of SEQ ID NO. 9, a polypeptide with about 95% homology, about 96%, about 97%, about 98%, about 99% homology with SEQ ID NO. 9.

In another embodiment, a polypeptide of SEQ ID NO. 10, a polypeptide with about 95% homology, about 96%, about 97%, about 98%, or about 99% homology with SEQ ID NO. 10 can be used. In another embodiment, a polypeptide of SEQ ID NO. 11, a polypeptide with about 95% homology, about 96%, about 97%, about 98%, or about 99% homology with SEQ ID NO. 11 can be used.

```
Human Progranulin SEQ ID NO: 2
MWTLVSWVALTAGLVAGTRCPDGQFCPVACCLDPGGASYSCCRP
LLDKWPTTLSRHLGGPCQVDAHCSAGHSCIFTVSGTSSCCPFPEAVACGDGHHCCPRG
FHCSADGRSCFQRSGNNSVGAIQCPDSQFECPDFSTCCVMVDGSWGCCPMPQASCCED
RVHCCPHGAFCDLVHTRCITPTGTHPLAKKLPAQRTNRAVALSSSVMCPDARSRCPDG
STCCELPSGKYGCCPMPNATCCSDHLHCCPQDTVCDLIQSKCLSKENATTDLLTKLPA
HTVGDVKCDMEVSCPDGYTCCRLQSGAWGCCPFTQAVCCEDHIHCCPAGFTCDTQKGT
CEQGPHQVPWMEKAPAHLSLPDPQALKRDVPCDNVSSCPSSDTCCQLTSGEWGCCPIP
EAVCCSDHQHCCPQGYTCVAEGQCQRGSEIVAGLEKMPARRASLSHPRDIGCDQHTSC
PVGQTCCPSLGGSWACCQLPHAVCCEDRQHCCPAGYTCNVKARSCEKEVVSAQPATFL
ARSPHVGVKDVECGEGHFCHDNQTCCRDNRQGWACCPYRQGVCCADRRHCCPAGFRCA
ARGTKCLRREAPRWDAPLRDPALRQLL
Human Progranulin DNA SEQ ID NO: 1
      1 ggcgagagga agcagggagg agagtgattt gagtagaaaa gaaacacagc attccaggct
    61 ggccccacct ctatattgat aagtagccaa tgggagcggg tagccctgat ccctggccaa
  121 tggaaactga ggtaggcggg tcatcgcgct ggggtctgta gtctgagcgc tacccggttg
  181 ctgctgccca aggaccgcgg agtcggacgc aggcagacca tgtggaccct ggtgagctgg
  241 gtggccttaa cagcagggct ggtggctgga acgcggtgcc cagatggtca gttctgccct
  301 gtggcctgct gcctggaccc cggaggagcc agctacagct gctgccgtcc ccttctggac
  361 aaatggccca caacactgag caggcatctg ggtggcccct gccaggttga tgcccactgc
  421 tctgccggcc actcctgcat ctttaccgtc tcagggactt ccagttgctg ccccttccca
  481 gaggccgtgg catgcgggga tggccatcac tgctgcccac ggggcttcca ctgcagtgca
  541 gacgggcgat cctgcttcca aagatcaggt aacaactccg tgggtgccat ccagtgccct
  601 gatagtcagt tcgaatgccc ggacttctcc acgtgctgtg ttatggtcga tggctcctgg
  661 gggtgctgcc ccatgcccca ggcttcctgc tgtgaagaca gggtgcactg ctgtccgcac
  721 ggtgccttct gcgacctggt tcacacccgc tgcatcacac ccacgggcac ccaccccctg
  781 gcaaagaagc tccctgccca gaggactaac agggcagtgg ccttgtccag ctcggtcatg
  841 tgtccggacg cacggtcccg gtgccctgat ggttctacct gctgtgagct gcccagtggg
  901 aagtatggct gctgcccaat gcccaacgcc acctgctgct ccgatcacct gcactgctgc
  961 ccccaagaca ctgtgtgtga cctgatccag agtaagtgcc tctccaagga gaacgctacc
1021 acggacctcc tcactaagct gcctgcgcac acagtggggg atgtgaaatg tgacatggag
1081 gtgagctgcc cagatggcta tacctgctgc cgtctacagt cgggggcctg gggctgctgc
1141 ccttttaccc aggctgtgtg ctgtgaggac cacatacact gctgtcccgc ggggtttacg
1201 tgtgacacgc agaagggtac ctgtgaacag gggccccacc aggtgccctg gatggagaag
1261 gccccagctc acctcagcct gccagaccca caagccttga agagagatgt cccctgtgat
1321 aatgtcagca gctgtccctc ctccgatacc tgctgccaac tcacgtctgg ggagtggggc
1381 tgctgtccaa tcccagaggc tgtctgctgc tcggaccacc agcactgctg cccccagggc
1441 tacacgtgtg tagctgaggg gcagtgtcag cgaggaagcg agatcgtggc tggactggag
1501 aagatgcctg cccgccgggc ttccttatcc caccccagag acatcggctg tgaccagcac
1561 accagctgcc cggtggggca gacctgctgc ccgagcctgg gtgggagctg ggcctgctgc
1621 cagttgcccc atgctgtgtg ctgcgaggat cgccagcact gctgcccggc tggctacacc
1681 tgcaacgtga aggctcgatc ctgcgagaag gaagtggtct ctgcccagcc tgccaccttc
1741 ctggcccgta gccctcacgt gggtgtgaag gacgtggagt gtggggaagg acacttctgc
1801 catgataacc agacctgctg ccgagacaac cgacagggct gggcctgctg tccctaccgc
1861 cagggcgtct gttgtgctga tcggcgccac tgctgtcctg ctggcttccg ctgcgcagcc
1921 aggggtacca agtgtttgcg cagggaggcc ccgcgctggg acgccccttt gagggaccca
1981 gccttgagac agctgctgtg agggacagta ctgaagactc tgcagccctc gggaccccac
2041 tcggagggtg ccctctgctc aggcctccct agcacctccc cctaaccaaa ttctccctgg
2101 accccattct gagctcccca tcaccatggg aggtggggcc tcaatctaag gccttccctg
2161 tcagaagggg gttgtggcaa aagccacatt acaagctgcc atcccctccc cgtttcagtg
2221 gaccctgtgg ccaggtgctt ttccctatcc acaggggtgt ttgtgtgtgt gcgcgtgtgc
2281 gtttcaataa agtttgtaca ctttcaaaaa aaaaaaaaaa aaa
Mouse Progranulin SEQ ID NO: 12
MWVLMSWLAFAAGLVAGTQCPDGQFCPVACCLDQGGANYSCCNP
LLDTWPRITSHHLDGSCQTHGHCPAGYSCLLTVSGTSSCCPFSKGVSCGDGYHCCPQG
FHCSADGKSCFQMSDNPLGAVQCPGSQFECPDSATCCIMVDGSWGCCPMPQASCCEDR
VHCCPHGASCDLVHTRCVSPTGTHTLLKKFPAQKTNRAVSLPFSVVCPDAKTQCPDDS
TCCELPTGKYGCCPMPNAICCSDHLHCCPQDTVCDLIQSKCLSKNYTTDLLTKLPGYP
VKEVKCDMEVSCPEGYTCCRLNTGAWGCCPFAKAVCCEDHIHCCPAGFQCHTEKGTCE
MGILQVPWMKKVIAPLRLPDPQILKSDTPCDDFTRCPTNNTCCKLNSGDWGCCPIPEA
VCCSDNQHCCPQGFTCLAQGYCQKGDTMVAGLEKIPARQTTPLQIGDIGCDQHTSCPV
GQTCCPSLKGSWACCQLPHAVCCEDRQHCCPAGYTCNVKARTCEKDVDFIQPPVLLTL
GPKVGNVECGEGHFCHDNQTCCKDSAGVWACCPYLKGVCCRDGRHCCPGGFHCSARGT
KCLRKKIPRWDMFLRDPVPRPLL
Mouse Progranulin DNA SEQ ID NO: 13
      1 gagatgcctc ccagggagcc cggaccccga cgcaggcaga ccatgtgggt cctgatgagc
    61 tggctggcct tcgcggcagg gctggtagcc ggaacacagt gtccagatgg gcagttctgc
  121 cctgttgcct gctgccttga ccagggagga gccaactaca gctgctgtaa ccctcttctg
  181 gacacatggc ctagaataac gagccatcat ctagatggct cctgccagac ccatggccac
  241 tgtcctgctg gctattcttg tcttctcact gtgtctggga cttccagctg ctgcccgttc
  301 tctaagggtg tgtcttgtgg tgatggctac cactgctgcc cccagggctt ccactgtagt
  361 gcagatggga aatcctgctt ccagatgtca gataacccct tgggtgctgt ccagtgtcct
  421 gggagccagt ttgaatgtcc tgactctgcc acctgctgca ttatggttga tggttcgtgg
  481 ggatgttgtc ccatgcccca ggcctcttgc tgtgaagaca gagtgcattg ctgtccccat
  541 ggggcctcct gtgacctggt tcacacacga tgcgtttcac ccacgggcac ccacacccta
  601 ctaaagaagt tccctgcaca aaagaccaac agggcagtgt ctttgccttt ttctgtcgtg
  661 tgccctgatg ctaagaccca gtgtcccgat gattctacct gctgtgagct acccactggg
  721 aagtatggct gctgtccaat gcccaatgcc atctgctgtt ccgaccacct gcactgctgc
  781 ccccaggaca ctgtatgtga cctgatccag agtaagtgcc tatccaagaa ctacaccacg
  841 gatctcctga ccaagctgcc tggataccca gtgaaggagg tgaagtgcga catggaggtg
  901 agctgccctg aaggatatac ctgctgccgc ctcaacactg gggcctgggg ctgctgtcca
  961 tttgccaagg ccgtgtgttg tgaggatcac attcattgct gcccggcagg gtttcagtgt
1021 cacacagaga aaggaacctg cgaaatgggt atcctccaag taccctggat gaagaaggtc
1081 atagcccccc tccgcctgcc agacccacag atcttgaaga gtgatacacc ttgtgatgac
1141 ttcactaggt gtcctacaaa caatacctgc tgcaaactca attctgggga ctggggctgc
1201 tgtcccatcc cagaggctgt ctgctgctca gacaaccagc attgctgccc tcagggcttc
1261 acatgtctgg ctcaggggta ctgtcagaag ggagacacaa tggtggctgg cctggagaag
1321 atacctgccc gccagacaac cccgctccaa attggagata tcggttgtga ccagcatacc
1381 agctgcccag tagggcaaac ctgctgccca agcctcaagg gaagttgggc ctgctgccag
1441 ctgccccatg ctgtgtgctg tgaggaccgg cagcactgtt gcccggccgg gtacacctgc
1501 aatgtgaagg cgaggacctg tgagaaggat gtcgatttta tccagcctcc cgtgctcctg
1561 accctcggcc ctaaggttgg gaatgtggag tgtggagaag ggcatttctg ccatgataac
1621 cagacctgtt gtaaagacag tgcaggagtc tgggcctgct gtccctacct aaagggtgtc
1681 tgctgtagag atggacgtca ctgttgcccc ggtggcttcc actgttcagc caggggaacc
1741 aagtgtttgc gaaagaagat tcctcgctgg gacatgtttt tgagggatcc ggtcccaaga
1801 ccgctactgt aaggaagggc tacagactta aggaactcca cagtcctggg aaccctgttc
1861 cgagggtacc cactactcag gcctccctag cgcctcctcc cctaacgtct ccccggccta
1921 ctcatcctga gtcaccctat caccatggga ggtggagcct caaactaaaa ccttctttta
1981 tggaaagaag gctgtggcca aaagccccgt atcaaactgc catttcttcc ggtttctgtg
2041 gaccttgtgg ccaggtgctc ttcccgagcc acaggtgttc tgtgagcttg cttgtgtgtg
2101 tgtgcgcgtg tgcgtgtgtt gctccaataa agtttgtaca ctttc
SEQ ID NO: 3 hGrnA
DVKCDMEVS-CPDGYTCCRLQSGAWGCCPFTQAVCCEDHIHCCPAGFTCDTQKGTC
EQ
SEQ ID NO: 4 hGrnB
-VMCPDARSRCPDGSTCCELPSGKYGCCPMPNATCCSDHLHCCPQDTVCDLIQSKCL
S
SEQ ID NO: 5 hGrnC
-VPCDNVSS-CPSSDTCCQLTSGEWGCCPIPEAVCCSDHQHCCPQGYTCVAEGQ-CQ
SEQ ID NO: 6 hGrnD
DIGCDQHTS-CPVGQTCCPSLGGSWACCQLPHAVCCEDRQHCCPAGYTCNVKARSC
E
SEQ ID NO: 7 hGrnE
DVECGEGHF-CHDNQTCCRDNRQGWACCPYRQGVCCADRRHCCPAGFRCAARGTK
CL
SEQ ID NO: 8 hGrnF
AIQCPDSQFECPDFSTCCVMVDGSWGCCPMPQASCCEDRVHCCPHGAFCDLVHTRCI
SEQ ID NO: 9 hGrnG
GGPCQVDAH-CSAGHSCIFTVSGTSSCCPFPEAVACGDGHHCCPRGFHCSADGRSCF
SEQ ID NO: 10 grn D
AMDIGCDQHTS-CPVGQTCCPSLGGSWACCQLPHAVCCEDRQHCCPAGYTCNVKAR
SCE-KLAAALEHHHHHH
SEQ ID NO: 11 grn F
AMAIQCPDSQFECPDFSTCCVMVDGSWGCCPMPQASCCEDRVHCCPHGAFCDLVHT
RCI-KLAAALEHHHHHH
```

As is well known to those skilled in the art, altering any non-critical amino acid of a protein by conservative substitution should not significantly alter the activity of that protein because the side-chain of the amino acid which is used to replace the natural amino acid should be able to form similar bonds and contacts as the side chain of the amino acid which has been replaced.

Non-conservative substitutions are possible provided that these do not excessively affect the activity of the progranulin polypeptide and/or reduce its effectiveness.

As is well-known in the art, a “conservative substitution” of an amino acid or a “conservative substitution variant” of a polypeptide refers to an amino acid substitution which maintains: 1) the structure of the backbone of the polypeptide (e.g., a beta sheet or alpha-helical structure); 2) the charge or hydrophobicity of the amino acid; and 3) the bulkiness of the side chain or any one or more of these characteristics. More specifically, the well-known terminologies “hydrophilic residues” relate to serine or threonine. “Hydrophobic residues” relate to leucine, isoleucine, phenylalanine, valine or alanine. “Positively charged residues” relate to lysine, arginine or histidine. “Negatively charged residues” relate to aspartic acid or glutamic acid. Residues having “bulky side chains” relate to phenylalanine, tryptophan or tyrosine.

The terminology “conservative amino acid substitutions” is well-known in the art, and relates to substitution of a particular amino acid by one having a similar characteristic (e.g., similar charge or hydrophobicity or similar bulkiness). Examples include aspartic acid for glutamic acid, or isoleucine for leucine. A list of illustrative conservative amino acid substitutions is given in TABLE 1. A conservative substitution variant will 1) have only conservative amino acid substitutions relative to the parent sequence, 2) will have at least 90% sequence identity with respect to the parent sequence, preferably at least 95% identity, 96% identity, 97% identity, 98% identity or 99% or greater identity; and 3) will retain activity of the progranulin polypeptide. In this regard, any conservative substitution variant of the above-described polypeptide sequences is contemplated in accordance with this invention. Such variants are considered to be a progranulin.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D- |
| | Gln |
| Aspartic Acid | D-Asp, D-Asn, Asn, Glu, D-Glu, Gln, D- |
| | Gln |
| Cysteine | D-Cys, S-Me-Cys, Met, D-Met, Thr, D- |
| | Thr |
| Glutamine | D-Gln, Asn, D-Asn, Glu, D-Glu, Asp, D- |
| | Asp |
| Glutamic Acid | D-Glu, D-Asp, Asp, Asn, D-Asn, Gln, D- |
| | Gln |
| Glycine | Ala, D-Ala, Pro, D-Pro, Aib, β-Ala |
| Isoleucine | D-Ile, Val, D-Val, Leu, D-Leu, Met, D- |
| | Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D- |
| | Trp |
| Proline | D-Pro |
| Serine | D-Ser, Thr, D-Thr, allo-Thr, L-Cys, D- |
| | Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D- |
| | Trp |
| Valine | D-Val, Leu, D-Leu, Ile, D-Ile, Met, D- |
| | Met |

In one illustrative aspect, the neurodegenerative disease can include, but is not limited to, Parkinson's disease and the parkinsonisms including progressive supranuclear palsy, Alzheimer's disease, and motor neuron disease (e.g., amyotrophic lateral sclerosis); or any other neurodegenerative disease mediated by a modification of progranulin expression.

In another embodiment, a pharmaceutical composition is provided. The pharmaceutical composition comprises therapeutically effective amounts of a progranulin and a pharmaceutically acceptable carrier, wherein the therapeutically effective amounts comprise amounts of a progranulin effective for increasing the activity or expression of neprilysin in the entorhinal cortex or frontal cortex of a patient with neurodegenerative disease or in the brain of an individual without a neurodegenerative disease. In another embodiment, the pharmaceutical composition comprises therapeutically effective amounts of a progranulin and a pharmaceutically acceptable carrier, wherein the therapeutically effective amounts comprise amounts effective for reducing microglia in the brain of a patient with a neurodegenerative disease.

The unitary daily dosage of the composition comprising a progranulin polypeptide can vary significantly depending on the patient condition, the disease state being treated, the route of administration of progranulin and tissue distribution, and the possibility of co-usage of other therapeutic treatments. The effective amount of a progranulin to be administered to the patient is based on body surface area, patient weight, physician assessment of patient condition, and the like. In one illustrative embodiment, an effective dose of a progranulin can range from about 1 ng/kg of patient body weight to about 1 mg/kg of patient body weight, more preferably from about 1 ng/kg of patient body weight to about 500 ng/kg of patient body weight, and most preferably from about 1 ng/kg of patient body weight to about 100 ng/kg of patient body weight.

In another illustrative embodiment, an effective dose of a progranulin polypeptide can range from about 1 pg/kg of patient body weight to about 1 mg/kg of patient body weight. In various illustrative embodiments, an effective dose can range from about 1 pg/kg of patient body weight to about 500 ng/kg of patient body weight, from about 500 pg/kg of patient body weight to about 500 ng/kg of patient body weight, from about 1 ng/kg of patient body weight to about 500 ng/kg of patient body weight, from about 100 ng/kg of patient body weight to about 500 ng/kg of patient body weight, and from about 1 ng/kg of patient body weight to about 100 ng/kg of patient body weight.

In another illustrative embodiment, an effective dose of a progranulin polypeptide can range from about 1 μg/kg of patient body weight to about 1 mg/kg of patient body weight. In various illustrative embodiments, an effective dose can range from about 1 μg/kg of patient body weight to about 500 μg/kg of patient body weight, from about 500 ng/kg of patient body weight to about 500 μg/kg of patient body weight, from about 1 μg/kg of patient body weight to about 500 μg/kg of patient body weight, from about 0.1 μg/kg of patient body weight to about 5 μg/kg of patient body weight, from about 0.1 μg/kg of patient body weight to about 10 μg/kg of patient body weight, and from about 0.1 μg/kg of patient body weight to about 100 μg/kg of patient body weight.

The composition comprising a progranulin is preferably administered to the patient parenterally, e.g., intradermally, subcutaneously, intramuscularly, intraperitoneally, intravenously, intraventricularly, intrathecally, intracerebrally or intracordally (spinal). Alternatively, the progranulin composition may be administered to the patient by other medically useful processes, and any effective dose and suitable therapeutic dosage form, including prolonged or sustained release dosage forms, can be used. Administration can be by injection. The composition comprising progranulin can also be delivered using a slow pump.

Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form in accordance with this invention can be in the form of a reconstitutable lyophilizate comprising a dose of a composition comprising a progranulin. In one aspect of the present embodiment, any of a number of prolonged or sustained release dosage forms known in the art can be administered such as, for example, the biodegradable carbohydrate matrices described in U.S. Pat. Nos. 4,713,249; 5,266,333; and 5,417,982, the disclosures of which are incorporated herein by reference.

In an illustrative embodiment pharmaceutical formulations for general use with progranulins for parenteral administration comprising: a) a pharmaceutically active amount of a progranulin; b) a pharmaceutically acceptable pH buffering agent to provide a pH in the range of about pH 4.5 to about pH 9; c) an ionic strength modifying agent in the concentration range of about 0 to about 250 millimolar; and d) water soluble viscosity modifying agent in the concentration range of about 0.5% to about 7% total formula weight are described or any combinations of a), b), c) and d).

In various illustrative embodiments, the pH buffering agents for use in the compositions and methods herein described are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, Cacodylate, and MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and non-ionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. It is preferred that non-acidic viscosity enhancing agents, such as a neutral or basic agent be employed in order to facilitate achieving the desired pH of the formulation. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed above. In one preferred embodiment, the viscosity modulating agent is cellulose that has been modified such as by etherification or esterification.

In various illustrative embodiments, progranulin compositions are provided that may comprise all or portions of progranulin polypeptides, alone or in combination with at least one other agent, such as an excipient and/or a stabilizing compound and/or a solubilizing agent, and may be administered in any sterile, biocompatible pharmaceutical carrier, including, but not limited to, saline, buffered saline, dextrose, glucose, and water. Suitable excipients are carbohydrate or protein fillers such as sugars, including lactose, sucrose, mannitol, or sorbitol; starch from corn, wheat, rice, potato, etc; celluloses such as methyl cellulose, hydroxypropylmethyl-cellulose, or sodium carboxymethylcellulose; and gums including arabic and tragacanth; and proteins such as gelatin and collagen. Suitable disintegrating or solubilizing agents include agar, alginic acid or a salt thereof such as sodium alginate.

In illustrative embodiments, progranulin polypeptides can be administered to a patient alone, or in combination with other agents, drugs or hormones or in pharmaceutical compositions mixed with excipient(s) or other pharmaceutically acceptable carriers. In one embodiment, the pharmaceutically acceptable carrier is pharmaceutically inert. In another embodiment, a progranulin may be administered alone to a patient suffering from a neurological disease or to an individual without a neurodegenerative disease.

Any effective regimen for administering the composition comprising a progranulin can be used. For example, the composition comprising progranulin can be administered as a single dose, or the composition comprising progranulin can be divided and administered as a multiple-dose daily regimen. Further, a staggered regimen, for example, one to three days per week can be used as an alternative to daily treatment, and for the purposes of this invention such intermittent or staggered daily regimen is considered to be equivalent to every day treatment and within the scope of this invention. In one embodiment, the patient, or the individual without a neurodegenerative disease, is treated with multiple injections of the composition comprising a progranulin. In another embodiment, the patient is injected multiple times (e.g., about 2 up to about 50 times) with the composition comprising a progranulin, for example, at 12-72 hour intervals or at 48-72 hour intervals. Additional injections of the composition comprising a progranulin can be administered to the patient at an interval of days or months after the initial injections(s) and the additional injections prevent recurrence of disease. Alternatively, the initial injection(s) of the composition comprising a progranulin may prevent recurrence of disease. In another illustrative embodiment, the composition comprising a progranulin can be administered to an individual without a neurodegenerative disease to prevent the neurodegenerative disease.

In another illustrative embodiment, patients with a neurodegenerative disease can be treated by administering to the patient a composition comprising an effector (e.g., a DNA encoding a therapeutic molecule, such as DNA's encoding progranulin or portions of progranulin), or combinations of effectors, that modify progranulin expression, wherein treatment of the patient with the composition comprising the effector that modifies progranulin expression increases the activity or expression of neprilysin in the entorhinal cortex or frontal cortex of a patient with neurodegenerative disease, or in any part of the brain of a patient without neurodegenerative disease.

In another embodiment, patients with a neurodegenerative disease can be treated by administering to the patient a composition comprising an effector, or combinations of effectors, that modify progranulin expression, wherein treatment of the patient with the composition comprising the effector that modifies progranulin expression reduces microglia in the brain of the patient with neurodegenerative disease.

In another embodiment, the composition comprising an effector can be administered to an individual without a neurodegenerative disease to prevent the neurodegenerative disease.

In any of the embodiments described herein involving administration of a progranulin effector, any of the formulations, treatment regimens, dose regimens, etc., can be used.

In yet another embodiment, a pharmaceutical composition is provided. The pharmaceutical composition comprises therapeutically effective amounts of an effector that modifies progranulin expression, and a pharmaceutically acceptable carrier, wherein the therapeutically effective amounts comprise amounts capable of increasing the activity or expression of neprilysin in the entorhinal cortex or frontal cortex of a patient with neurodegenerative disease.

In another embodiment, a method is provided for increasing the activity or expression of neprilysin in the entorhinal cortex or frontal cortex of a patient with neurodegenerative disease. The method comprises the step of administering to a patient with a neurodegenerative disease a therapeutically effective amount of an effector that modifies progranulin expression, wherein the amount of the effector is effective to increase the activity or expression of neprilysin in the entorhinal cortex or frontal cortex of a patient with neurodegenerative disease.

In another embodiment, a method is provided for reducing microglia in the brain of a patient with neurodegenerative disease. The method comprises the step of administering to the patient with the neurodegenerative disease a therapeutically effective amount of an effector that modifies progranulin expression, wherein the amount of effector is effective to reduce microglia in the brain of the patient with neurodegenerative disease.

In the above-described embodiments, the composition comprising an effector can be administered to an individual without a neurodegenerative disease to prevent the neurodegenerative disease.

As used herein, an effector that modifies progranulin expression means a nucleic acid (e.g., a DNA, a cDNA, or an mRNA) that increases progranulin expression in target cells (e.g., SEQ ID NO. 1). As used herein target cells comprise neuronal cells. The unitary daily dosage of the composition comprising the effector that modifies progranulin expression can vary significantly depending on the patient condition, the disease state being treated, the molecular weight of the effector, its route of administration and tissue distribution, and the possibility of co-usage of other therapeutic treatments. The effective amount to be administered to the patient is based on body surface area, patient weight, and physician assessment of patient condition. In one illustrative embodiment, an effective dose of the effector can range from about 1 ng/kg of patient body weight to about 1 mg/kg of patient body weight, more preferably from about 1 ng/kg of patient body weight to about 500 ng/kg of patient body weight, and most preferably from about 1 ng/kg of patient body weight to about 100 ng/kg of patient body weight.

In another illustrative embodiment, an effective dose of the effector can range from about 1 pg/kg of patient body weight to about 1 mg/kg of patient body weight. In various illustrative embodiments, an effective dose can range from about 1 pg/kg of patient body weight to about 500 ng/kg of patient body weight, from about 500 pg/kg of patient body weight to about 500 ng/kg of patient body weight, from about 1 ng/kg of patient body weight to about 500 ng/kg of patient body weight, from about 100 ng/kg of patient body weight to about 500 ng/kg of patient body weight, and from about 1 ng/kg of patient body weight to about 100 ng/kg of patient body weight.

In another illustrative embodiment, an effective dose of the effector can range from about 1 million effector molecules per 70 kg patient to about 1 billion effector molecules per 70 kg patient. In various illustrative embodiments, an effective dose can range from about 1 million effector molecules per 70 kg patient to about 500 million effector molecules per 70 kg patient, from about 200,000 effector molecules per 70 kg patient to about 200 million effector molecules per 70 kg patient, from about 1 million effector molecules per 70 kg patient to about 200 million effector molecules per 70 kg patient.

The composition comprising the effector that modifies progranulin expression is preferably administered to the patient parenterally, e.g., intradermally, subcutaneously, intramuscularly, intraperitoneally, intravenously, intraventricularly, intrathecally, intracerebrally or intracordally (spinal). Alternatively, the composition comprising the effector that modifies progranulin expression may be administered to the patient by other medically useful processes, and any effective dose and suitable therapeutic dosage form, including prolonged release dosage forms, can be used. Administration can be accomplished by injection.

The composition comprising the effector that modifies progranulin expression is preferably injected parenterally and such injections can be intradermal injections, intraperitoneal injections, subcutaneous injections, intramuscular injections, intravenous injections, intraventricular injections, intrathecal injections, intracerebral injections or intracordal injections (spinal). The composition comprising the effector that modifies progranulin expression can also be delivered using a slow pump. Additionally, suitable routes may, for example, include oral or transmucosal administration. Therapeutic administration of an effector that modifies progranulin expression intracellularly can also be accomplished as described below.

Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form in accordance with this invention can be in the form of a reconstitutable lyophilizate comprising a dose of a composition comprising an effector that modifies progranulin expression. In one aspect of the present embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference.

Any effective regimen for administering the composition comprising the effector that modifies progranulin expression can be used. For example, the composition comprising the effector that modifies progranulin expression can be administered as a single dose, or the composition comprising the effector that modifies progranulin expression can be administered in multiple doses. Further, a staggered regimen, for example, one to three days per week can be used as an alternative to daily treatment, and for the purposes of this invention such intermittent or staggered daily regimen is considered to be equivalent to every day treatment and within the scope of this invention. In one embodiment, the patient is treated with one or more injections of the composition comprising the effector that modifies progranulin expression. In another embodiment, the patient is injected multiple times (e.g., about 2 up to about 50 times) with the composition comprising the effector that modifies progranulin expression, for example, at 12-72 hour intervals or at 48-72 hour intervals. Additional injections of the composition comprising the effector that modifies progranulin expression can be administered to the patient at an interval of days or months after the initial injections(s) and the additional injections prevent recurrence of disease. Alternatively, the initial one or more injection(s) of the composition comprising the effector that modifies progranulin expression may prevent recurrence of disease. In another embodiment, the composition comprising an effector can be administered to an individual without a neurodegenerative disease to prevent the neurodegenerative disease.

In various illustrative embodiments, the presently described compositions comprise an isolated and purified nucleic acid sequence encoding progranulin or a portion thereof. Methods of purifying nucleic acids are well-known to those skilled in the art. In one embodiment, the sequence is operatively linked to regulatory sequences directing expression of progranulin. In further embodiments, the sequence is operably linked to a heterologous or homologous promoter. In still further embodiments, the sequence is contained within a vector. In some embodiments, the vector is within a host cell (e.g., a neuronal cell). In one embodiment, the vector is a lentivirus vector (e.g., Invitrogen, Carlsbad, Calif.).

As used herein, the term vector is used in reference to nucleic acid molecules that transfer DNA or mRNA segment(s) to cells of the patient. The vector contains the nucleic acid sequence and appropriate nucleic acid sequences necessary for the expression of the operably linked nucleic acid coding sequence in the patient. A vector is capable of expressing a nucleic acid molecule inserted into the vector and, of producing a polypeptide or protein, or portion thereof. Nucleic acid sequences necessary for expression usually include a promoter, an operator (optional), and a ribosome binding site, often along with other sequences such as enhancers, and termination and polyadenylation signals.

If a cell is used for delivery of the nucleic acid, the nucleic acid may be introduced into the cell by transducing, transfecting, microinjecting, or electroporating, the cell with the nucleic acid. A delivery cell may be transformed, transduced, or transfected (e.g., by calcium phosphate-DNA co-precipitation, DEAE-dextran-mediated transfection, polybrene-mediated transfection, electroporation, microinjection, liposome fusion, lipofection, protoplast fusion, retroviral infection, biolistics, etc.) by exogenous or heterologous nucleic acids when such nucleic acids have been introduced inside the cell. Transforming DNA, for example, may or may not be integrated (covalently linked) with chromosomal DNA making up the genome of the delivery cell. In mammalian cells for example, transforming DNA may be maintained on an episomal element, such as a plasmid. In a eukaryotic cell, a stably transformed cell is one in which the transforming DNA has become integrated into a chromosome so that it is inherited by daughter cells through chromosome replication.

As used herein, the effector that modifies progranulin expression can comprise a progranulin nucleic acid and the progranulin nucleic acid comprises a complete progranulin coding sequence, a portion thereof, or a homologous sequence as described herein.

In another illustrative embodiment, a progranulin nucleic acid can be incorporated into a vector and administered to a patient by any protocol known in the art such as those described in U.S. Pat. Nos. 6,333,194, 7,105,342 and 7,112,668, incorporated herein by reference. In illustrative embodiments, a progranulin nucleic acid, can be introduced either in vitro into a cell extracted from an organ of the patient wherein the modified cell then being reintroduced into the body, or directly in vivo into the appropriate tissue or using a targeted vector-progranulin nucleic acid construct. In various illustrative embodiments, the progranulin nucleic acid can be introduced into a cell or an organ using, for example, a viral vector, a retroviral vector, or non-viral methods, such as transfection, injection of naked DNA, electroporation, sonoporation, a gene gun (e.g., by shooting DNA coated gold particles into cells using high pressure gas), synthetic oligomers, lipoplexes, polyplexes, virosomes, or dendrimers.

In one embodiment where cells or organs are treated, the progranulin nucleic acid can be introduced into a cell or organ using a viral vector. The viral vector can be any viral vector known in the art. For example, the viral vector can be an adenovirus vector, a lentivirus vector, a retrovirus vector, an adeno-associated virus vector, a herpesvirus vector, a modified herpesvirus vector, and the like. In one embodiment, the vector is a lentivirus vector (e.g., Invitrogen, Carlsbad, Calif.). In another illustrative embodiment where cells are transfected, the progranulin nucleic acid can be introduced into a cell by direct DNA transfection (lipofection, calcium phosphate transfection, DEAE-dextran, electroporation, and the like).

In various illustrative embodiments, the progranulin nucleic acid can be, for example, a DNA molecule, an RNA molecule, a cDNA molecule, or an expression construct comprising a progranulin nucleic acid.

The progranulin nucleic acids described herein can be prepared or isolated by any conventional means typically used to prepare or isolate nucleic acids and include the nucleic acids of SEQ ID. No. (1) and (13). For example, DNA and RNA molecules can be chemically synthesized using commercially available reagents and synthesizers by methods that are known in the art. The progranulin nucleic acids described herein can be purified by any conventional means typically used in the art to purify nucleic acids. For example, the progranulin nucleic acids can be purified using electrophoretic methods and nucleic acid purification kits known in the art (e.g. Quiagen kits). Progranulin nucleic acids suitable for delivery using a viral vector or for introduction into a cell by direct DNA transfection can also be prepared using any of the recombinant methods known in the art.

Nucleic acids having modified internucleoside linkages can also be used in the methods and compositions herein described. Nucleic acids containing modified internucleoside linkages can be synthesized using reagents and methods that are known in the art, for example, methods for synthesizing nucleic acids containing phosphonate, phosphorothioate, phosphorodithioate, phosphoramidate methoxyethyl phosphoramidate, formacetal, thioformacetal, diisopropylsilyl, acetamidate, carbamate, dimethylene-sulfide (—CH.sub.2-S—CH.sub.2-), dimethylene-sulfoxide (—CH.sub.2-SO—CH.sub.2-), dimethylene-sulfone (—CH.sub.2-SO.sub.2-CH.sub.2-), 2′-O-alkyl, and 2′-deoxy-2′-fluorophosphorothioate internucleoside linkages.

Modified progranulin sequences, i.e. sequences that differ from the sequence encoding native progranulin, are also encompassed by the invention, so long as the modified sequence still encodes a protein that exhibits the biological activity of the native progranulin at a greater or lesser level of activity. These modified progranulin sequences include modifications caused by point mutations, modifications due to the degeneracy of the genetic code or naturally occurring allelic variants, and further modifications that are introduced by genetic engineering, to produce recombinant progranulin nucleic acids.

Progranulin nucleic acids include nucleic acids with 95% homology to SEQ ID Nos. 1 or 13 or to nucleic acids which hybridize under highly stringent conditions to the complement of the DNA coding sequence for a progranulin SEQ ID Nos. 1 or 13. As used herein, the term hybridization is used in reference to the pairing of complementary nucleic acids. Hybridization and the strength of hybridization (e.g., the strength of the association between the nucleic acids) is impacted by such factors as the degree of complementary between the nucleic acids, stringency of the conditions involved, the Tₘ (melting temperature) of the formed hybrid, and the G:C ratio within the nucleic acids. As used herein the term stringency is used in reference to the conditions of temperature, ionic strength, and the presence of other compounds such as organic solvents, under which nucleic acid hybridizations are conducted.

In an illustrative example, highly stringent conditions can mean hybridization at 65° C. in 5×SSPE and 50% formamide, and washing at 65° C. in 0.5×SSPE. In another illustrative example, highly stringent conditions can mean hybridization at 55° C. in a hybridization buffer consisting of 50% formamide (vol/vol); 10% dextran sulfate; 1×Denhardt's solution; 20 mM sodium phosphate, pH 6.5; 5×SSC; and 200 μg of salmon sperm DNA per ml of hybridization buffer for 18 to 24 hours, and washing four times (5 min each time) with 2×SSC; 1% SDS at room temperature and then washing for 15 min at 50-55° C. with 0.1×SSC. In another illustrative example conditions for high stringency hybridization are described in Sambrook et al., Molecular Cloning: A Laboratory Manual, 3rd Edition, Cold Spring Harbor Laboratory Press, (2001), incorporated herein by reference. In some illustrative aspects, hybridization occurs along the full-length of the nucleic acid.

Detection of highly stringent hybridization in the context of the present invention indicates strong structural similarity or structural homology (e.g., nucleotide structure, base composition, arrangement or order) to, e.g., the nucleic acids provided herein.

Also included are nucleic acid molecules having about 80%, about 85%, about 90%, about 95%, 96%, 97%, 98%, and 99% homology to the DNA coding sequence for a progranulin SEQ ID No. 1 or 13. As used herein, the percent homology between two sequences is equivalent to the percent identity between the sequences. Determination of percent identity or homology between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software (Altschul et al., 1990). In some embodiments, the percent homology or identity can be determined along the full-length of the nucleic acid.

As used herein, the term complementary refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have complementary sequences. The complementary sequences can be DNA or RNA sequences. The complementary DNA or RNA sequences are referred to as a complement. Complementarity may be partial, in which only some of the nucleic acids' bases are matched according to the base pairing rules. Or, there may be complete or total complementarity between the nucleic acids.

In illustrative embodiments, the neurodegenerative disease is mediated by an environmental insult to the patient. As used herein, a neurodegenerative disease mediated by an environmental insult to the patient means a disease that is caused by an environmental insult and is not caused by a heritable mutation of the progranulin gene that modifies progranulin expression. A heritable mutation is a permanent mutation in a patient's DNA that may be transmitted to the patient's offspring. These illustrative embodiments are however not meant to exclude the influence of allelic variants of modifier genes, that are, for example, involved in the metabolism of the neurotoxin, that render an individual more or less sensitive to neurodegenerative disease development. As used herein these modifier genes can modify the course of disease development.

The neurodegenerative disease mediated by environmental insult to the patient may be a sporadic disease linked to environmental factors that cause neuronal cell death directly or indirectly by modifying gene expression. In various other illustrative embodiments, the environmental insult is derived from the patient's diet or is the result of endogenous synthesis, or both. In one illustrative embodiment, the environmental insult causes synthesis of a compound that causes a detrimental effect in vivo. The neuronal cell death may occur by any variety of means including, but not limited to, excitotoxicity or oxidative stress. For example, various means by which environmental toxins lead to neuronal cell death are described in U.S. Patent Application Publication No. 2006-0252705, which is hereby incorporated by reference.

In another illustrative embodiment, the neurodegenerative disease state is mediated by an excitotoxin. Excitotoxins are a class of substances that damage neurons through overactivation of receptors, for example, receptors for the excitatory neurotransmitter glutamate, leading to neuronal cell death. Examples of excitotoxins include excitatory amino acids, which can produce lesions in the central nervous system. Additional examples of excitotoxins include, but are not limited to, sterol glucoside, including beta-sitosterol-beta-D-glucoside and cholesterol glucoside, methionine sulfoximine, and other substances known in the art to induce neuro-excitotoxic reactions in a patient. In one illustrative embodiment, the excitotoxin is a sterol glycoside. In further illustrative embodiments, the sterol glycoside is selected from the group consisting of beta-sitosterol-beta-D-glucoside and cholesterol glucoside, or analogs or derivatives thereof.

In one illustrative embodiment, the neurodegenerative disease is selected from the group consisting of Parkinson's disease, Alzheimer's disease, and ALS. Neurological diseases, including Alzheimer's disease, Parkinson's disease, and ALS, generally result in behavioral deficits that can be observed clinically. These diseases target populations of neurons leading to neuropathological and behavioral symptoms. Alzheimer's disease involves the death of neurons of various regions of the cerebral cortex and the hippocampus and results in the loss of cognitive functions such as memory and learning. Parkinson's disease results in degeneration of portions of the nigral-striatal system. Initial stages involve the loss of terminal projections of dopamine-containing neurons from the substantia nigra. In turn, the neuron cell bodies in the substantia nigra die, impacting motor control and leading to tremor and gait disturbances.

An example of a motor neuron disease is amyotrophic lateral sclerosis (ALS). ALS primarily involves the loss of spinal and cortical motor neurons, leading to increasing paralysis and eventually death. Early symptoms of ALS include but are not limited to, footdrop or weakness in a patient's legs, feet, or ankles, hand weakness or clumsiness, muscle cramps and twitching in the arms, shoulders, and tongue. ALS generally affects chewing, swallowing, speaking, and breathing, and eventually leads to paralysis of the muscles required to perform these functions. A review of various neurological diseases is set forth in Shaw et al., Neuroscience and Biobehavioral Reviews, 27: 493 (2003), which is hereby incorporated by reference. The method and compositions of the present invention can be used for both human clinical medicine and veterinary medicine applications. The methods and compositions described herein may be used alone, or in combination with other methods or compositions.

In another illustrative embodiment, patients with a neurodegenerative disease can be treated by administering to the patient a composition comprising an effector (e.g., a DNA encoding a therapeutic molecule), or combinations of effectors, that modifies progranulin expression, wherein treatment of the patient with the composition comprising the effector that modifies progranulin expression reduces the symptoms of the neurological disease in the patient. Any of the above embodiments using effectors that mediate progranulin expression are applicable to this embodiment.

In yet another embodiment, a pharmaceutical composition is provided. The pharmaceutical composition comprises therapeutically effective amounts of an effector that modifies progranulin expression, and a pharmaceutically acceptable carrier, wherein the therapeutically effective amounts comprise amounts capable of reducing the symptoms of a neurodegenerative disease in a patient with neurodegenerative disease or preventing the symptoms of a neurodegenerative disease in an individual without neurodegenerative disease. Any of the above embodiments (e.g., formulation embodiments, dose regimen embodiments, treatment regimen embodiments, etc.) using effectors that mediate progranulin expression are applicable to this embodiment.

### Example 1

### Animals

Lentiviral Infusion was performed on either wild type C57BL/6 mice or the Tg2576 mouse model of Alzheimer's Disease. The Tg2576 mouse model of Alzheimer's Disease expresses the Swedish mutation of APP (APP_{K67ON,M671L}) at high levels under the control of the hamster prion protein promoter. These mice generate high levels of brain Aβ, and develop a progressive, age-related deposition in the form of amyloid plaques in the hippocampus, entorhinal and frontal cortex, similar to those seen in humans. To assess the influence of progranulin on the development of these plaques, as well as its effects on neprilysin expression, eight month old mice were treated, via unilateral intra-hippocampal infusion, with a recombinant lentiviral vector encoding either green fluorescent protein (GFP) or progranulin (PGRN). Animals were then sacrificed by perfusion at 12 months of age. Tissues were then analyzed for progranulin expression, plaque burden and neprilysin expression.

Animals:

Studies with the Tg2576 mice used 20-25 g female mice. Animals were housed in a temperature-controlled environment with a 12 h light/dark cycle and ad libitum access to standard chow and water. Procedures used in this study were approved by the Mayo Foundation Institutional Animal Care and Use Committee (IACUC).

Mice were sacrificed by transcardial perfusion of 0.9% saline, the brains removed and post-fixed in 4% paraformaldehyde for histochemical and immunohistochemical analysis. Symmetrical 20 μm-thick coronal sections were cut on a cryostat and stored in a Millonigs solution until used.

### Example 2

### Microscopy

Microscopy and all photomicrographs from mouse sections were captured using a Motic B5 Professional Series 3.0 (Motic Instruments Inc., Richmond, Canada) camera and Zeiss Axiovert Epiflorescence 2000 microscope. Data were analyzed using Motic B5 Professional, Motic Images Advanced 3.0 and Zeiss Axiovert Zoom Axiovision 3.1 with AxioCam HRM.

### Example 3

### Lentiviral Vector

The progranulin-expressing lentiviral vector (Invitrogen Corporation (Carlsbad, Calif.)) titer was determined to be 1×10⁸ TU/mL by the blasticidin resistance assay. The lentivirus was stored in cryovials frozen at −80° C. until the day of injection.

### Example 4

### Progranulin Immunohistochemistry

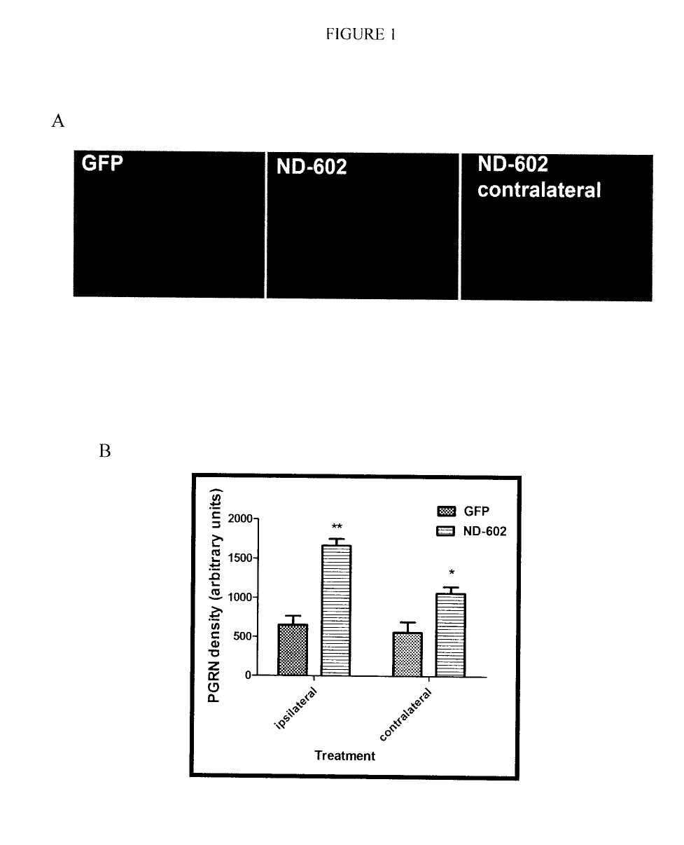

Free-floating sections were washed 3×10 minutes, with gentle agitation at room temperature, in 1×TBS-triton (20 mM Tris, pH=7.4 and 0.9% NaCl, 0.3% triton X-100). Sections were then blocked for 30 minutes, in 5% donkey serum in TBS-t, with gentle agitation at room temperature. Following this, sections were exposed to sheep anti-progranulin antibody (R& D Systems, 1:1000 dilution in 2% donkey serum), initially for 2 hours at room temperature followed by an overnight incubation at 4° C., with gentle agitation. The following day, the primary antibody was removed and sections were washed, 3×10 minutes, with gentle agitation at room temperature in 1×TBS-t. Sections were then exposed to secondary antibody (Donkey anti-Sheep 1:500, Invitrogen: Molecular Probes Cat #A-21097 in 1% donkey serum/TBSt) for 4 hours at RT with gentle agitation. Secondary antibody was removed by washing 3×10 minutes, with gentle agitation at room temperature, in 1×TBS-t. Sections were then mounted onto slides using Fluoromount G (Electron Microscopy Sciences, Cat #17984-25), cover slipped with 24×60 mm No. 0 cover slips (Electron Microscopy Sciences, Cat #63751-01) and imaged. See FIG. 1 for results.

### Example 5

### Progranulin Immunohistochemistry

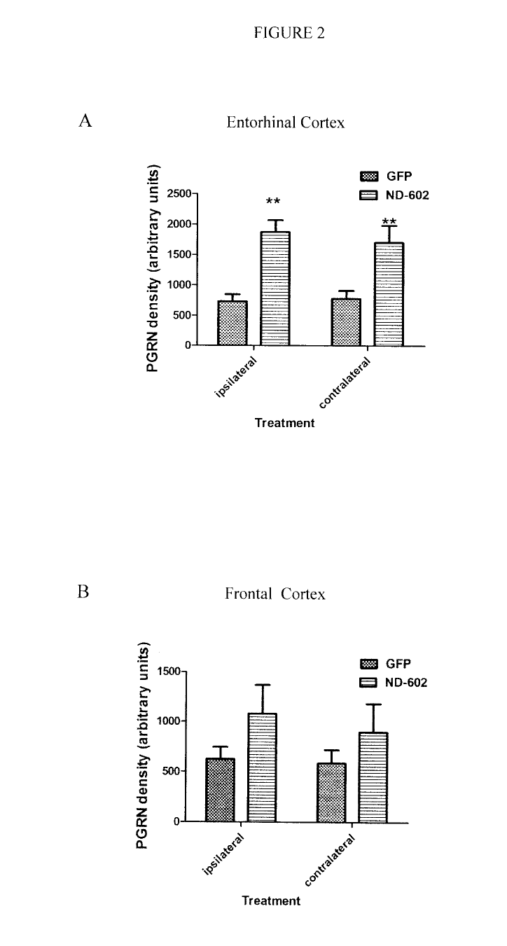

The results presented in FIG. 2 were obtained using the same protocols as described in Example 4.

### Example 6

### Neprilysin Immunoreactivity

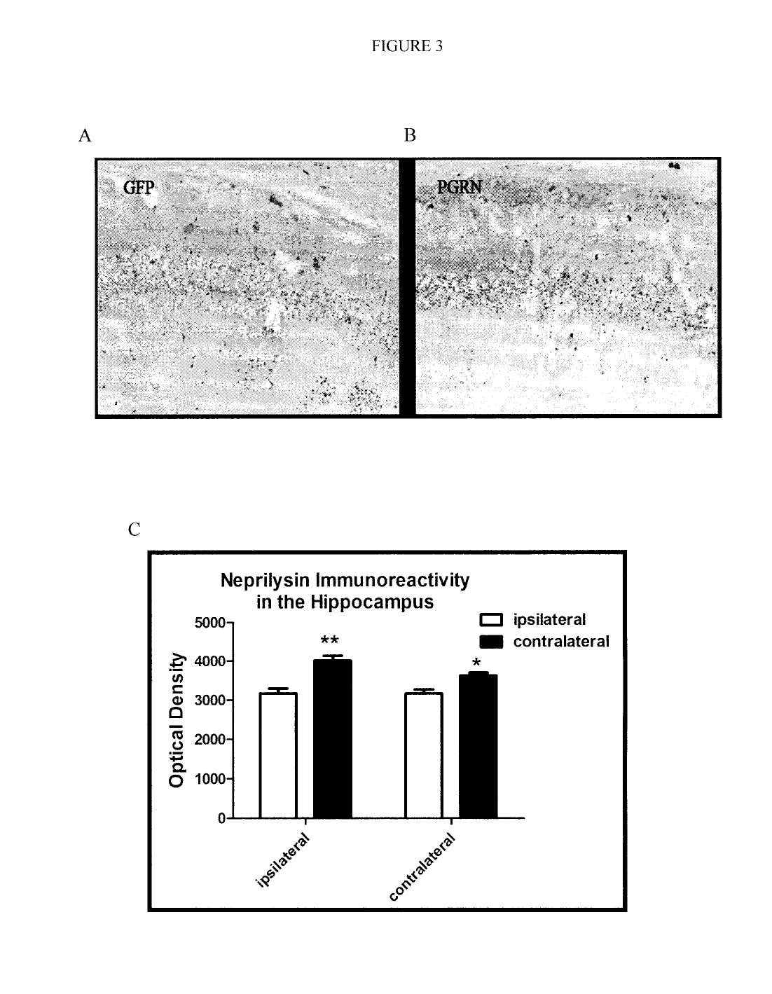

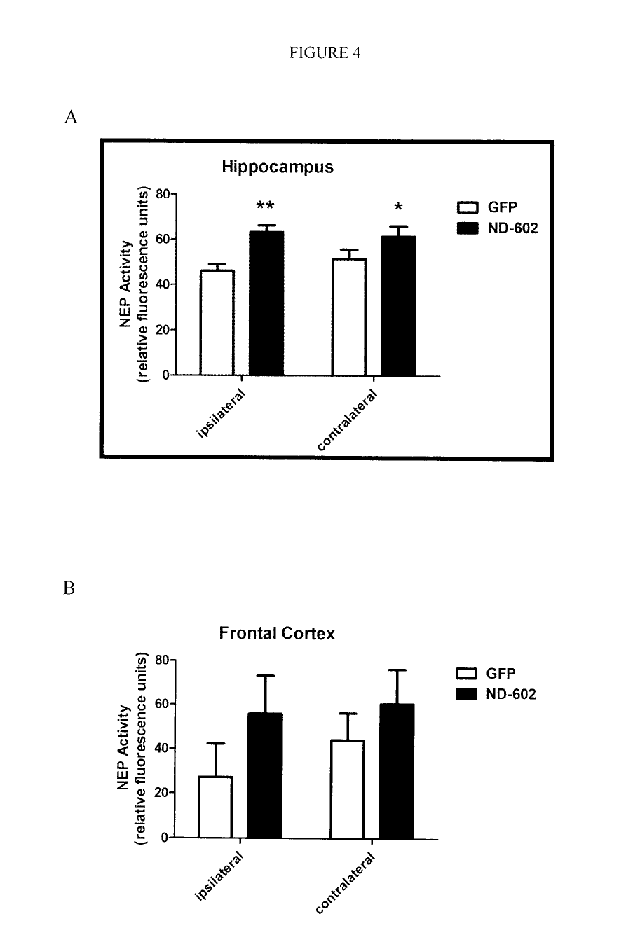

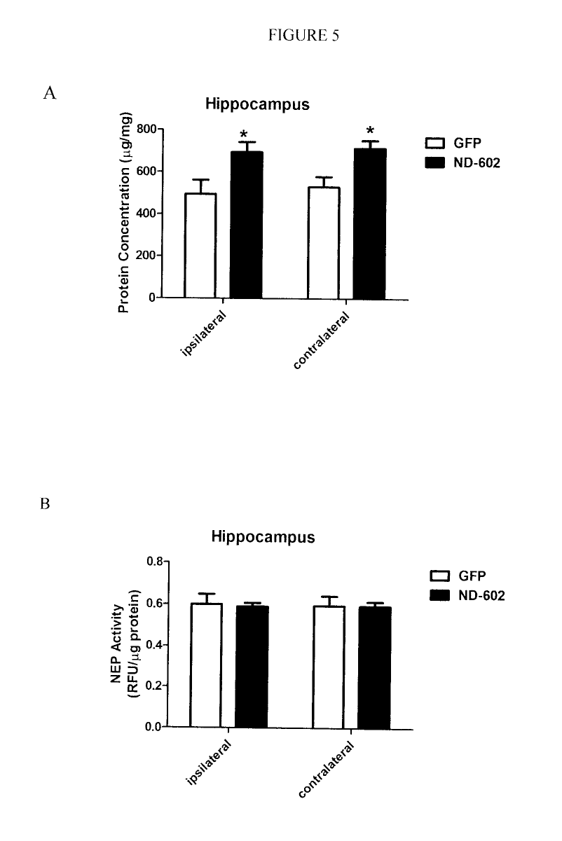

Neprilysin is considered to be the rate-limiting enzyme in the degradation of amyloid-β. The effects of progranulin on both neprilysin protein expression (FIG. 3) and Neprilysin activity (see below FIGS. 4 and 5) were determined. All manipulations of tissue were as described above for the progranulin immunohistochemistry. Here, sections were blocked with 5% goat serum and the primary antibody used was a rabbit anti-neprilysin (1:500 dilution in 2% goat serum, Millipore Cat#AB5458). The secondary antibody was a goat anti-rabbit 1:500 dilution (Invitrogen: Molecular Probes Cat #A-11037).

### Example 7

### Neprilysin Activity

This assay was carried out on wild type C57/BL6 mice that had been subjected to lentiviral infusion. Colony reared female C57/BL6 mice (3 months old) were purchased from Charles River (St. Hyacinth Qc.). All animal procedures were approved by the Animal Care Committee of the Atlantic Veterinary College at the University of Prince Edward Island, and were identical to those described previously for viral infusion into Tg2576 mice. Animals were sacrificed by decapitation at one month following viral infusion and the brains were blocked to include either the entire dorsal hippocampus, or frontal regions (n=6 animals). The results are shown in FIGS. 4 and 5.

DAGNPG (N-dansyl-Alpha-Gly-D-nitro-Phe-Gly, modeled after enkephalins with an aromatic moiety in the P′1 position and a short residue in the P′2 position) is an internally quenched substrate of NEP. NEP degrades DAGNPG to DAG (Km=45 μM, V=0.65 μmol/mg protein/min), and the liberated dansyl group can be excited at a wavelength of 342 nm and emits at 562 nm. ACE can also degrade DAGNPG, therefore an ACE inhibitor is necessary to resolve NEP activity (must pre-incubate for 10 minutes, using at least 0.5 μM captopril). NEP has optimal activity in 50 mM Tris HCL, pH 7.4 and inhibited by 20 nM Thiorphan (Ki=3 nM).

Tissue Lysis:

Weighed tissue was placed in ice cold 50 mM Tris-HCl, pH 7.4 (3 mg/ml). The tissue was then homogenized on ice by 30 strokes in a homogenizer (VWR, Model VBI 25) set at position 1. Further disruption of tissue was accomplished by sonication on ice for 15 seconds (Fisher Scientific Sonic Dismembrator Model 100) at setting #5, using a microprobe. The homogenate was transferred to a sterile microcentrifuge tube and centrifuged at 10,000×g at 4° C. for 30 minutes. The supernatant was then transferred to another sterile centrifuge tube and the same centrifugation repeated. Two hundred μl of this supernatant was then used to assay neprilysin activity.

Enzyme Assay:

Captopril (Sigma, Cat #C40402, final concentration 0.5 μM), with or without a Thiorphan (Sigma, Cat #T6031, final concentration of 20 nM) were added to the sample in a final volume of 200 μl. The reaction mixture was incubated at 37° C. for 30 minutes, after which 1 mM DAGNPG (N-dansyl-Alpha-Gly-D-nitro-Phe-Gly, Sigma, Cat #D2155, final concentration 50 μM) was added the sample, mixed and incubated at 37° C. for an additional 2 hours. The reaction was stopped by denaturing the enzyme activity by heating to 100° C. for 5 minutes. The resulting mixture was centrifuged at 5,000×g for 5 minutes at room temperature. One hundred and seventy five μl of the resulting supernatant were removed and diluted into 400 μl of 50 mM Tris-HCL, pH 7.4, and mixed (vortex 10 seconds). Two hundred microliters of the mixture were then aliquoted into a to a 96-well plate and emission at 550 nM read using a Spectra max M2, (Molecular Devices) plate reader (excitation at 355 nM).

### Example 8

### Neprilysin Activity and Expression

The neprilysin activity assays were performed as described in Example 7 (see FIGS. 4 and 5). A bicinchoninic acid (BCA) based protein assay was performed using a commercially available protein assay kit (Thermo Scientific, cat #232225) according to the manufacturer's instructions, using bovine serum albumin as a standard (see FIG. 5).

### Example 9

### Administration of Progranulin Effector

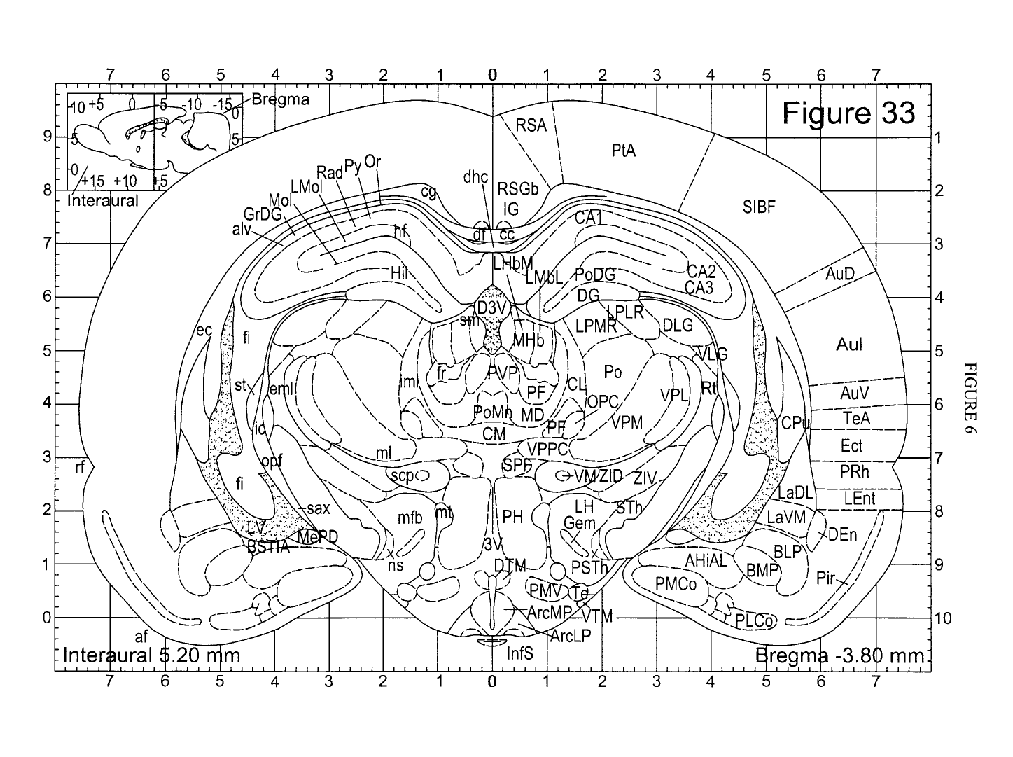

Animals were anaesthetized using isoflurane (1%) and placed in a Kopf stereotaxic frame. For hippocampal transduction, either GFP or PGRN lentiviral vector was injected unilaterally into the left hippocampus (A.P.—1.7, M.L.—15 1.5, D.V.—2.3) (2 μl/site) at a rate of 0.25 μl/minute via an infusion cannula connected by polyethylene tubing (50 PE) to a 50 μl Hamilton syringe driven by a Harvard Pump. Following infusion, the vector was permitted to diffuse away from the cannula for four minutes before withdrawal (FIG. 6).

### Example 10

### Reduction of Plaque Burden by Progranulin Effector

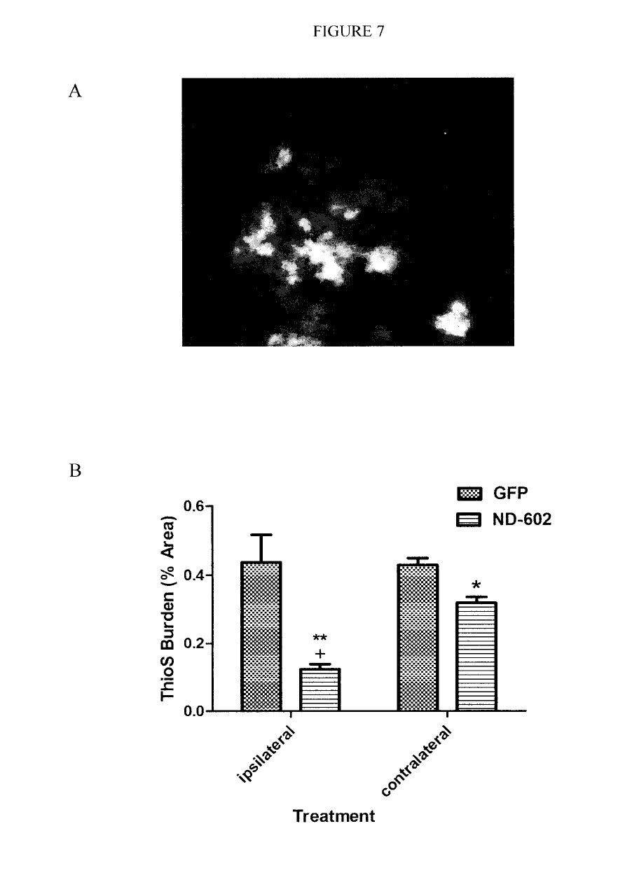

To detect amyloid deposits sections were rinsed in deionized water for 5 minutes then exposed in the dark to a freshly prepared 1% aqueous solution of Thioflavine S (Sigma cat #T1892) for 20 minutes at room temperature. The stain was differentiated for 5 minutes in a 70% ethanol:water solution. Following this, ethanol was removed by 2×5 second rinses in deionized water. Sections were then mounted for imaging as described above in the progranulin IHC protocol. The results are shown in FIG. 7.

### Example 11

### Reduction of Plaque Burden by Progranulin Effector

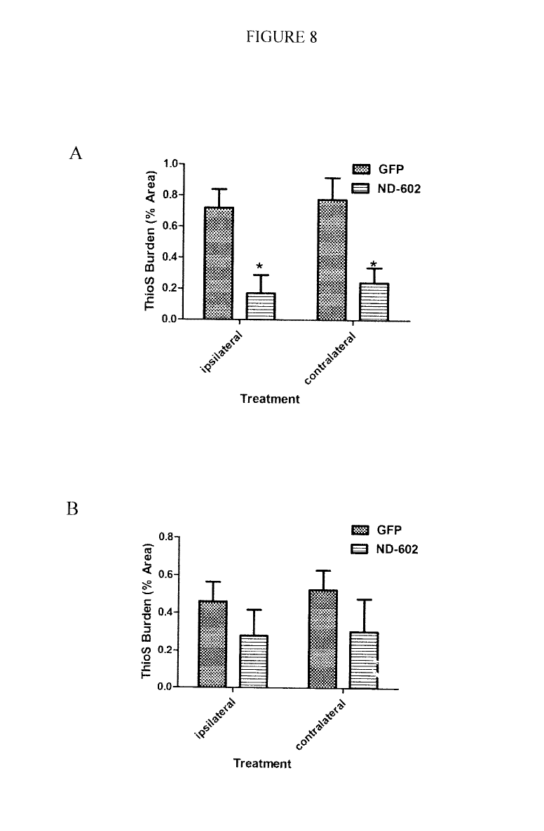

The assays were performed as described in Example 10. The results are shown in FIG. 8.

### Example 12

### Reduction of Microglia by Progranulin Effector

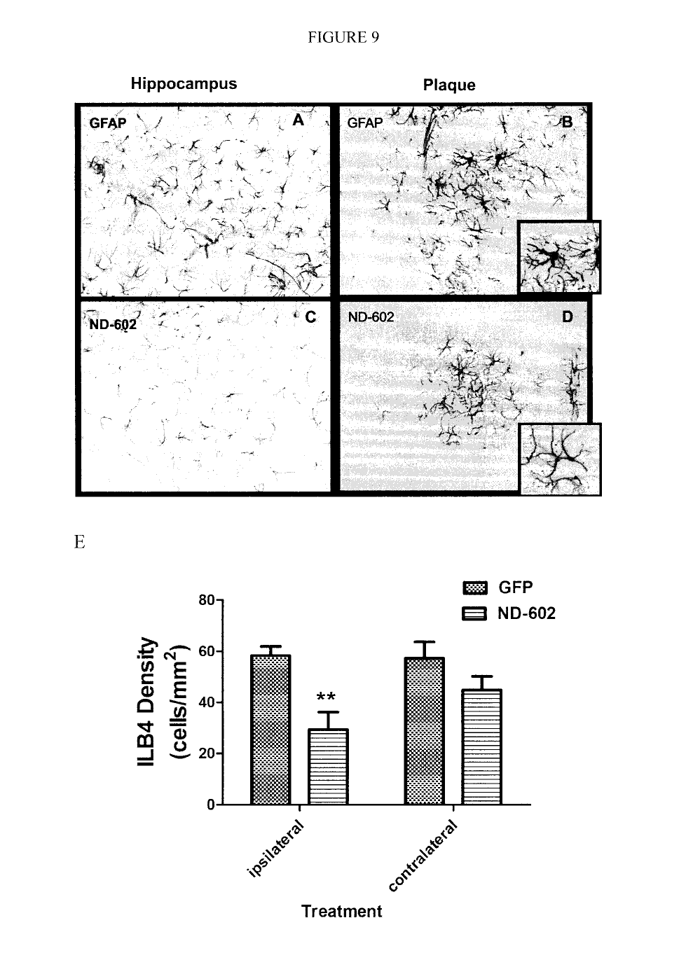

Isolectin B₄ from Griffonia Simplificolia binds avidly to microglial cells especially activated microglia. The effects of progranulin treatment on the neuroinflammatory response to the developing neuropathology in Tg2576 mice was evaluated (see FIG. 9). Free-floating sections were washed 3×10 minutes, with gentle agitation at room temperature, in 1×TBS-t. Sections were then blocked for 30 minutes, in 5% goat serum in TBS-t, with gentle agitation at room temperature. Following this sections were exposed to FITC conjugated ILB₄ (1:100 dilution in 3% goat serum, Vector labs cat #FL1201), overnight at 4° C. with gentle agitation. The following day the unbound ILB₄-FITC was removed by washing, 3×10 minutes, with gentle agitation at room temperature, in 1×TBS-t. Sections were then mounted onto slides using Fluoromount G (Electron Microscopy Sciences, Cat #17984-25), cover slipped with 24×60 mm No. 0 cover slips (Electron Microscopy Sciences, Cat #63751-01) and imaged.

Quantitative Analysis:

Densitometric quantitation of Progranulin IHC (FIGS. 1, 2) was accomplished by imaging the entire hippocampus in, and regions of entorhinal and frontal cortex present in 4 coronal sections per animal, using a computer-assisted image analysis system and Zeiss Axiovision 4.7 image analysis software (n=12). The same analysis was applied to the assessment of neprilysin IHC (FIG. 3). Plaque burden (FIGS. 7 & 8) in hippocampus, entorhinal cortex and frontal cortex was determined measuring the by the % of total area occupied by thioflavine S positive elements in each region using the Axiovision software to analyze images obtained using a 20× objective.

For determination of numbers of ILB₄ positive microglia (FIG. 9), 4 coronal sections containing hippocampus were assessed per animal (n=12). For neprilysin activity determination, activity determined with Thiorphan was subtracted from that obtained without Thiorphan. For all analyses, the investigator was blinded to treatment condition.

Statistical Analysis:

Data were analyzed using two way analysis of variance. Where significant F-values were obtained, planned pair-wise comparisons were made using Newman-Keuls post hoc test. Differences were considered statistically significant when p<0.05.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to,” and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

1.-23. (canceled)

24. A method for increasing the activity or expression of neprilysin in the frontal cortex or entorhinal cortex of a patient with neurodegenerative disease, the method comprising the step of
administering to the patient a composition comprising a progranulin polypeptide wherein the activity or expression of neprilysin in the frontal cortex or entorhinal cortex of the patient is increased.

25. The method of claim 24 wherein the amount of the progranulin polypeptide administered to the patient is in the range of about 1 ng/kg of patient body weight to about 1 mg/kg of patient body weight.

26. The method of claim 25 wherein the amount of the progranulin polypeptide administered to the patient is in the range of about 1 ng/kg of patient body weight to about 500 ng/kg of patient body weight.

27. The method of claim 25 wherein the amount of the progranulin polypeptide administered to the patient is in the range of about 1 ng/kg of patient body weight to about 100 ng/kg of patient body weight.

28. The method of claim 24 wherein the composition comprising the progranulin polypeptide is adapted for parenteral administration.

29. The method of claim 28 wherein the route of parenteral administration is selected from the group consisting of intradermally, subcutaneously, intramuscularly, intraperitoneally, intravenously, intraventricularly, intrathecally, intracerebrally, and intracordally.

30. The method of claim 24 wherein the neurodegenerative disease is Alzheimer's disease.

31. The method of claim 24 wherein the progranulin polypeptide has at least 95% homology with SEQ ID NO: 2.

32. The method of claim 24 wherein the neprilysin reduces plaque burden.

33. A method for increasing the activity or expression of neprilysin in the frontal cortex or entorhinal cortex of a patient with neurodegenerative disease, the method comprising the step of
administering to the patient a composition comprising an effector that modifies progranulin expression wherein the activity or expression of neprilysin in the frontal cortex or entorhinal cortex of the patient is increased.

34. The method of claim 33 wherein the amount of the effector administered to the patient is in the range of about 1 ng/kg of patient body weight to about 1 mg/kg of patient body weight.

35. The method of claim 34 wherein the amount of the effector administered to the patient is in the range of about 1 ng/kg of patient body weight to about 500 ng/kg of patient body weight.

36. The method of claim 34 wherein the amount of the effector administered to the patient is in the range of about 1 ng/kg of patient body weight to about 100 ng/kg of patient body weight.

37. The method of claim 33 wherein the composition comprising the effector is adapted for parenteral administration.

38. The method of claim 37 wherein the route of parenteral administration is selected from the group consisting of intradermally, subcutaneously, intramuscularly, intraperitoneally, intravenously, intraventricularly, intrathecally, intracerebrally, and intracordally.

39. The method of claim 33 wherein the neurodegenerative disease is Alzheimer's disease.

40. The method of claim 33 wherein the progranulin polypeptide has at least 95% homology with SEQ ID NO: 1.

41. The method of claim 33 wherein the neprilysin reduces plaque burden.

42. A method for increasing the activity or expression of neprilysin in the brain of an individual without a neurodegenerative disease to prevent the neurodegenerative disease, the method comprising the step of
administering to the individual a composition comprising a progranulin polypeptide wherein the neurodegenerative disease is prevented.

43. The method of claim 42 wherein the amount of the progranulin polypeptide administered to the patient is in the range of about 1 ng/kg of patient body weight to about 1 mg/kg of patient body weight.

44. The method of claim 43 wherein the amount of the progranulin polypeptide administered to the patient is in the range of about 1 ng/kg of patient body weight to about 500 ng/kg of patient body weight.

45. The method of claim 43 wherein the amount of the progranulin polypeptide administered to the patient is in the range of about 1 ng/kg of patient body weight to about 100 ng/kg of patient body weight.

46. The method of claim 42 wherein the composition comprising the progranulin polypeptide is adapted for parenteral administration.

47. The method of claim 46 wherein the route of parenteral administration is selected from the group consisting of intradermally, subcutaneously, intramuscularly, intraperitoneally, intravenously, intraventricularly, intrathecally, intracerebrally, and intracordally.

48. The method of claim 42 wherein the progranulin polypeptide has at least 95% homology with SEQ ID NO: 2.

49. The method of claim 42 wherein the neprilysin reduces plaque burden.

50. A method for increasing the activity or expression of neprilysin in the brain of an individual without a neurodegenerative disease to prevent the neurodegenerative disease, the method comprising the step of
administering to the individual a composition comprising an effector that modifies progranulin expression wherein the neurodegenerative disease is prevented.

51. The method of claim 50 wherein the amount of the effector administered to the patient is in the range of about 1 ng/kg of patient body weight to about 1 mg/kg of patient body weight.

52. The method of claim 51 wherein the amount of the effector administered to the patient is in the range of about 1 ng/kg of patient body weight to about 500 ng/kg of patient body weight.

53. The method of claim 51 wherein the amount of the effector administered to the patient is in the range of about 1 ng/kg of patient body weight to about 100 ng/kg of patient body weight.

54. The method of claim 50 wherein the composition comprising the effector is adapted for parenteral administration.

55. The method of claim 51 wherein the route of parenteral administration is selected from the group consisting of intradermally, subcutaneously, intramuscularly, intraperitoneally, intravenously, intraventricularly, intrathecally, intracerebrally, and intracordally.

56. The method of claim 50 wherein the progranulin polypeptide has at least 95% homology with SEQ ID NO: 1.

57. The method of claim 50 wherein the neprilysin reduces plaque burden.
